(19) United States
(12) Patent Application Publication
(10) Pub. No.: US 2024/0374289 A1
(43) Pub. Date: Nov. 14, 2024

(54) SYSTEM AND METHOD FOR OOCYTE RETRIEVAL

(71) Applicant: MAGNA MATER MEDICAL LTD., Tel-Aviv (IL)

(72) Inventor: Gil BACHAR, Tel-Aviv (IL)

(73) Assignee: MAGNA MATER MEDICAL LTD., Tel-Aviv (IL)

(21) Appl. No.: 18/690,737

(22) PCT Filed: Sep. 13, 2022
(86) PCT No.: PCT/IL2022/050991

§ 371 Date: Mar. 10, 2024

Related U.S. Application Data
(60) Provisional application No. 63/243,849, filed on Sep. 14, 2021.
(60) Provisional application No. 63/389,977, filed on Jul. 18, 2022.

(51) Int. Cl. A61B 17/435; A61B 17/00; G06T 7/00; G06V 10/774; G06V 20/50; G06V 20/69

(52) U.S. Cl. CPC A61B 17/435; G06T 7/0012; G06V 10/774; G06V 20/50; G06V 20/698; A61B 2017/00057; A61B 2217/005; G06T 2207/30024; G06V 2201/03

(57) ABSTRACT

A system for oocytes retrieval is disclosed. The system comprises: at least one camera; a holder configured to hold the camera and an oocytes retrieval tube such that a transparent portion of the oocytes retrieval tube is within the field of view (FOV) of the at least one camera; and a controller configured to: control the at least one camera to capture images of the transparent portion. The transparent portion is transparent to visible light.

### CROSS-REFERENCE TO RELATED PATENT APPLICATIONS

This patent application claims the benefit of priority from and is related to U.S. Provisional Patent Application Nos. 63/243,849, filed Sep. 14, 2021 and 63/389,977, filed Jul. 18, 2022. The contents of the above applications are all incorporated herein by reference as if fully set forth herein in its their entirety.

### FIELD OF THE INVENTION

The present invention relates generally to oocyte retrieval. More specifically, the present invention relates to systems and method to support decision making during oocyte retrieval process.

### BACKGROUND

Oocyte retrieval process is used as part of fertility problem solution or fertility preservation. To date, the most common oocyte retrieval process includes transvaginal needle insertion into the ovaries and suction of fluid from one or more follicles, the follicle fluid containing an oocyte (one oocyte per follicle).

Following suction, the follicle fluid with entrained oocytes flows from the needle out of a patient body and through a plastic tuning into a container. The container is transferred to an embryologist laboratory for examination, fertilization, freezing and other processes.

However, in this process, the physician conducting the oocyte retrieval process, has little to no knowledge as to whether an oocyte was actually obtained, and the quality, size and other parameters of the oocytes collected. Thus, and in order to ensure collection of a sufficient number of oocytes suitable for fertilization, freezing and the like, the above process is repeated several times, at different follicles, with multiple repetitions for each follicle.

These repetitions are painful to the patient and may raise the risk of infection during the process.

### SUMMARY

Some aspects of the invention are directed to a system for oocytes retrieval, comprising: at least one camera; a holder configured to hold the camera and an oocytes retrieval tube such that a transparent portion of the oocytes retrieval tube is within the field of view (FOV) of the at least one camera; and a controller configured to: control the at least one camera to capture images of the transparent portion.; In some embodiments the controller is further configured to control a suction unit, in fluid connection with the oocytes retrieval tube, based on an analysis of the captured images, oocytes retrieval tube. In some embodiments, the transparent portion is transparent to visible light, and the suction unit is configured to suction oocytes.

In some embodiments, controlling the suction unit comprises at least one of: terminating the suction, reinitiating the suction and changing the suction velocity. In some embodiments, the controller is further configured to identify oocytes in the captured images. In some embodiments, identifying the oocytes comprises identifying at least one of: number of oocytes, size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity.

In some embodiments, controlling the suction unit is based on the identification of the oocytes. In some embodiments, the controller is further configured to assign a score to at least some of the identified oocytes. In some embodiments, the score of an identified oocyte is based on at least one of: size of the identified oocyte, shape of the identified oocyte, morphology of the identified oocyte, cytoplasm of the identified oocyte, ooplasm characteristics of the identified oocyte, structure of the perivitelline space, corona radiata quality, zona pellucida, clarity and uniformity.

In some embodiments, the system includes the suction unit.

In some embodiments, the system includes a sorting unit for sorting the fluid flowing in the oocytes retrieval tube between at least two different containers. In some embodiments, controller is configured to control the sorting unit based on the identification. In some embodiments, the controller is configured to control the sorting unit based on analysis of the images captured by the camera.

In some embodiments, the system further includes a light source positioned to provide light to the transparent portion. In some embodiments, the camera comprises at least one sensor and at least one lens for magnifying objects in the transparent portion. In some embodiments, the at least one lens is a microscope lens configured to image the transparent portion such that is comprise at least 50% of the FOV. In some embodiments, the holder comprises an adjustment mechanism for adjusting the distance between the at least one lens and the objects in the transparent portion. In some embodiments, the controller is configured to adjust the adjustment mechanism based on images received form the at last one camera.

In some embodiments, the system further includes one or more containers for collecting the retrieve fluid.

Some additional aspects of the invention are directed to a method of oocytes retrieval, comprising: receiving one or more images of a fluid in a retrieval tube; and analyzing the one or more images for identifying one or more oocytes in the fluid.

In some embodiments, identifying the oocytes comprises identifying at least one of: number of oocytes, size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity.

In some embodiments, the method further comprises to assigning a score to at least some of the identified oocytes. In some embodiments, the score is given based on at least one of: size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity.

In some embodiments, the method further comprises sorting the fluid flowing in the oocytes retrieval tube between at least two different containers.

Some additional aspects of the invention are directed to a system for oocytes retrieval, comprising: at least one needle; at least one transparent tubing and at least one optical window, the optical window comprising at least one flat facet. In some embodiments, the at least one transparent tubing and the at least one optical window are made of materials having substantially the same refraction indexes. In some embodiments, the system further comprises a container cap.

Some additional aspects of the invention are directed to a method of classifying oocytes in a retrieved fluid, by at least one processor, said method comprising: receiving at least one image of the retrieved fluid from at least one camera; detecting one or more oocytes in the at least one image; extracting from the at least one image at least one feature related to the detected one or more oocytes; and applying a ML model on the extracted at least one feature to classify the one or more oocytes. In some embodiments, the ML model is trained to classify oocytes based on oocytes quality.

In some embodiments, training the ML model comprises: receiving a training dataset, comprising a plurality of images, each depicting at least one oocyte; receiving a set of quality labels, corresponding to the plurality of images; extracting from at least one image of the training dataset at least one respective feature of the depicted at least one oocyte; and using the set of quality labels as supervisory data for training the ML model to classify at least one depicted oocyte based on the extracted features.

In some embodiments, the at least one feature related to the oocyte is selected from: number of oocytes, size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 is a schematic illustration of a system for oocyte retrieval according to embodiments of the present invention;

FIG. 2 shows an illustration of dual imager configuration, according to embodiments of the present invention;

FIG. 3 shows another configuration of an imager according to embodiments of the present invention;

FIG. 4A is a schematic illustration of another system for oocyte retrieval according to embodiments of the present invention;

FIG. 4B shows an enlarged section in FIG. 4A showing a bath.

FIG. 5A shows an oocyte retrieval system according to embodiments of the present invention;

FIG. 5B shows the retrieval system from FIG. 5A positioned in a system for oocyte detection, according to embodiments of the present invention;

FIG. 5C. shows an optical window according to embodiments of the present invention;

FIG. 6 show examples of a separation mechanism according to embodiments of the present invention;

FIG. 7A is a flowchart of a method identifying oocytes in a retrieved fluid according to embodiments of the present invention;

FIG. 7B is a block diagram of a computer software system for classifying oocytes and of using a trained ML model according to embodiments of the present invention; and

FIG. 8 shows high-level block diagram of an exemplary computing device according to embodiments of the present invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a controller, a computing platform, a computing system, or other electronic computing device, that manipulates and/or transforms data represented as physical (e.g., electronic) quantities within the controller's registers and/or memories into other data similarly represented as physical quantities within the controller's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

A system and method according to embodiments of the invention may allow taking images of oocytes during the retrieval stage, analyzing the images and controlling the oocytes retrieval based on the analysis. Such a system may include a camera and holder configured to hold the camera and an oocytes retrieval tube. In some embodiments, the oocytes retrieval tube is insertable into a patient's body and/or connected to a needle insertable to the patient's body and\or connected to a catheter insertable to the patient's body. In some embodiments, the oocytes retrieval tube has at least one portion that is transparent to visible light or to a portion of the visible light spectrum or to infrared spectrum. In some embodiments, at least one transparent portion is covered with an optical window comprising at least one flat facet. In some embodiments, the system includes a controller to control the at least one camera to capture images of fluid flowing in the transparent portion, and control a suction unit, in fluid connection with the oocytes retrieval tube, based on an analysis of the captured images.

In some embodiments, the fluid flowing in the tube may include one or more oocytes, therefore, when passing in the transparent portion an image of the fluid may be captured by the camera. In some embodiments, the camera may include at least one sensor and at least one lens for magnifying objects (e.g., oocytes) in the transparent portion. In some embodiments, the controller may receive the magnified images of the fluid and may identify at least one oocyte in the images. In some embodiments, the identification may include number of oocytes and/or the quality of at least some of the oocytes. In some embodiments, the identification may include training and utilizing a machine learning (ML) model as discussed herein below.

In some embodiments, the controller may control the suction unit and/or control a sorting unit to retrieve and/or sort the retrieve liquid that comprises the oocytes. For example, the controller may control the suction unit to stop the suction in order to take an image of a fluid in the tube at substantially zero following velocity, if a real-time analysis of a stream of images, taken under from a flowing condition, indicated the existence of oocytes. In another example, the controller may control a sorting unit, comprising a plurality of controllable valves, to fill an oocytes container only with fluid containing oocytes and direct the rest of the fluid to other containers. In some embodiments, the controller may control the sorting unit to fill the oocytes container only with oocytes classified as having sufficient quality.

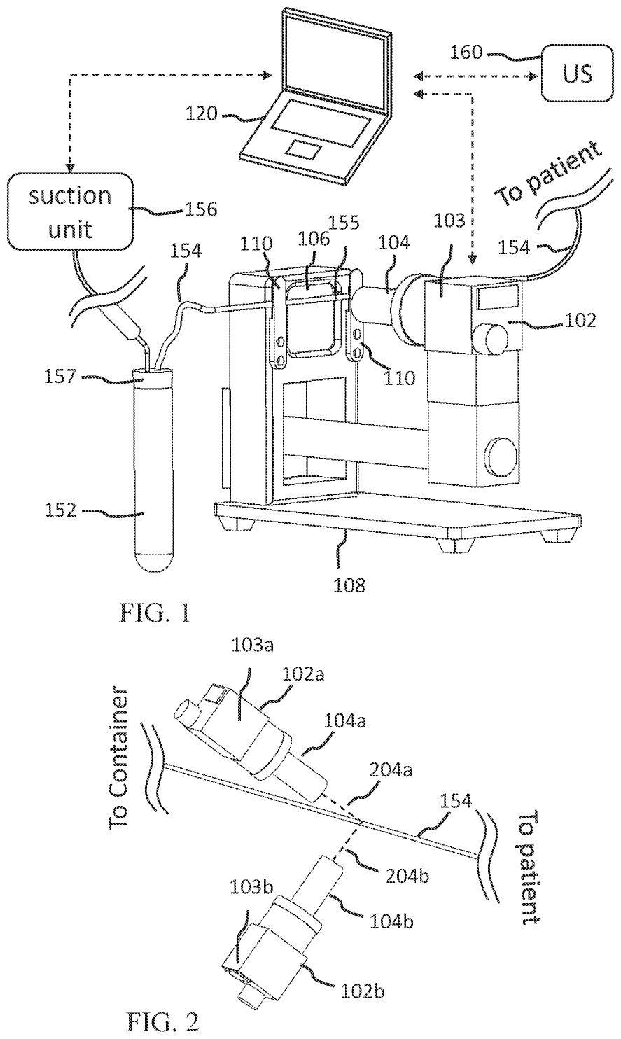

Reference is now made to FIG. 1 which is a schematic illustration of a system 100 for oocytes retrieval according to some embodiments. System 100 may be designed to image and detect cells flowing in a tube, and in particular oocytes. System 100 may be used during an operation for oocyte retrieval to support decision making. System 100 may detect oocytes in real time and indicate to the operator (e.g., surgeon, gynecologist, embryologist, nurse, etc.), not shown in the figure, on the progress of the operation.

System 100 may include at least one camera 102, 102a and/or 102b (illustrated also in FIG. 2), a holder 108 and a controller 120. Holder 108 is configured to hold camera 102 and an oocytes retrieval tube 154 such that a transparent portion 155 of the oocytes retrieval tube is within the field of view (FOV) of at least one camera 102 and within focus of at least one camera 102

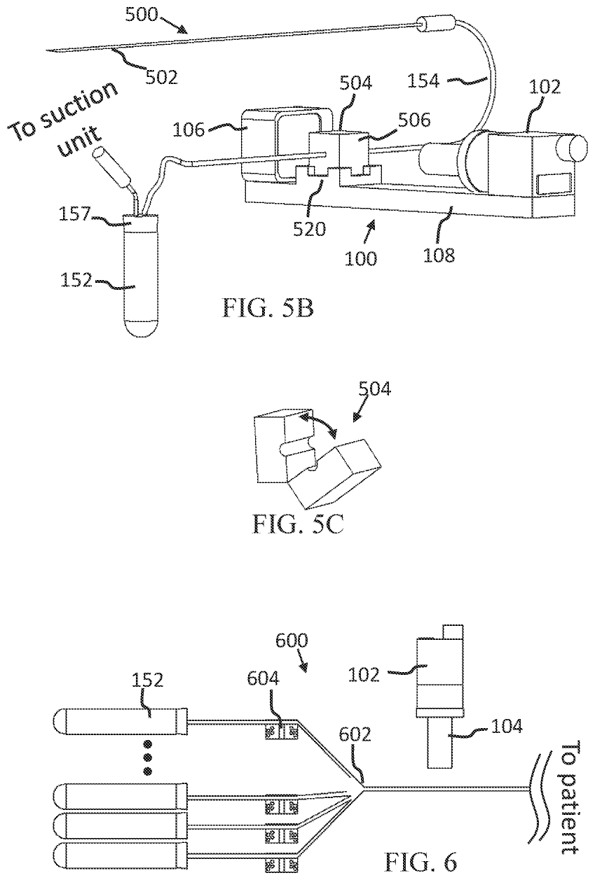

In some embodiments, oocytes retrieval tube 154 may be designed for transferring fluids coming from patient's body. For example, oocytes retrieval tube 154 may be insertable into a patient's body and/or may be connectable to a needle insertable into a patient's body (e.g., as seen in FIG. 5A). In some embodiments, at least one portion 155 is transparent to visible light or a portion of the visible light spectrum or to infrared wavelength. Oocytes retrieval tube 154 may be connected to a container 152 (e.g., test tube) via a container cap 157. Container cap 157 may allow fluid from tube 154 to flow into container 152. Container cap 157 may have an additional outlet 162, which may be connected to a suction unit 156 to create a vacuum in container 152 and draw fluids from tube 154.

In some embodiments, system 100 may or may not include suction unit 156. Suction unit 156 may be in fluid connection with oocytes retrieval tube 154, for suctioning oocytes.

In some embodiments, at least one camera 102, 102a and/or 102b (illustrated also in FIG. 2) is positioned such that transparent portion 155 is within the field of view (FOV) of the at least one camera 102. In some embodiment, controller 120 may be configured to control at least one camera 102, 102a and/or 102b to capture images of fluid flowing in transparent portion 155 and to control suction unit 156 based on an analysis of the captured images.

In some embodiments, container 152 may be connected to oocytes retrieval tube 154. In a non-limiting example, the entire oocytes retrieval tube 154 may be transparent to visible light or to a portion of the visible light spectrum. Tube 154 may continue toward patient's body. In some non-limiting example tube 154 may be connected to an aspiration needle (not seen in FIG. 1, an example for needle is shown in FIG. 5A) which is insertable into a patient's body for ovum pickup (OPU) as known in the art. In another non-limiting example tube 154 may be connected to an oocyte retrieval catheter demonstrated in a co-owned patent application. Container 152 may further be connected to suction unit 156 (e.g., pump, syringe or any other suction sources). Suction unit 156 may create vacuum force in container 152 which in turn pulls fluid in tube 154 from patient's body and toward contained 152. In an example, tube 154 may be connected to system 100, such that system 100 may image fluid flowing in tube 154 for oocyte identification, counting, grading, etc. In one embodiment, system 100 may be operated by a medical doctor, a nurse, other medical staff, the patient, etc. referred hereafter as “the operator”. In another embodiment, system 100 may be autonomous (i.e., self-operated without human intervention).

In some embodiments, at least one camera 102 may include at least one sensor 103 and at least one lens 104. In some embodiments, system 100 may further include a light source 106. In some embodiments, holder 108 may include one or more tubing holders 110. Tubing holders 110 may be used to position transparent part 155 within the FOV and\or focus range (DOF) of camera 102. In some embodiments, tubing holders 110 may assure the position of transparent part 155 relative to camera 102 within standard deviation of ±1 mm in all 3 axes (X-Y-Z) between repetitive positioning experiments. According to an example, camera 102 may be a digital camera (e.g., having a CMOS or CCD sensor 103), capable of high resolution (e.g., 0.5 Mega Pixel or more), high frame rate (e.g., more than 100 frames per second (FPS), more then 300 FPS, more than 1000 FPS or any value in between) and short exposure time (e.g., less than 100 microseconds (usec), less than 50 usec, less than 10 usec, or any value in between). High frame rate camera may assure that oocyte passing in tube 154 would be imaged by camera 102 at least once within the oocyte travel within camera 102 FOV. In some embodiments, camera 102 frame rate should be higher than Vo/HFOV, wherein HFOV is the horizontal field of view of camera 102 and Vo is the average speed of oocytes in tube 154. Short exposure time may assure that the oocyte images will not suffer from motion blur. In some embodiments, exposure time should be lower than Pxl/Vo, wherein Pxl is the size of pixel in sensor 103 and Vo is the average speed of an oocyte in tube 154. In some embodiments, sensor 103 may have a global shutter to avoid rolling shutter distortion effect. In some embodiments, camera 102 may be a monochromatic camera. In an example, camera 102 may be a color camera (e.g., red-green-blue). In some embodiments, at least some of the pixels of sensor 103 may include a light filter to absorb light only in a specified spectrum, for example, red spectrum (wavelength range), or only in deep-red spectrum or only in far-red spectrum or only in near infrared (NIR) spectrum. In some examples, at least some of the pixels of sensor 103 may include a light filter blocking light below 600 nanometer (nm) or below 630 nm or below 660 nm or below 700 nm or below 900 nm. In some embodiments, at least some of the pixels of sensor 103 may include a band pass light filter blocking light outside of range 600-750 nm outside of range 630 nm-700 nm or outside of range 900 nm-1100 nm. In some embodiments, a filter is tuned to a wavelength rage that may be defined such that if more than 90% of the power of light or more than 80% of the power of light from source 106 passing in the filter and captured by sensor 103 is originated in the specified spectrum (wavelength) range, In some embodiments, the filter is tuned to a spectrum range that may be defined such that the peak (maximal) power wavelength of light from source 106 passing in the filter and captured by sensor 103 is in the specified spectrum.

In some embodiments, at least one lens 104 is configured to image objects (e.g., oocytes) in the transparent portion on camera 102 sensor. In one example, at least one lens 104 is a microscope lens configured to magnify the objects in the transparent portion such that transparent portion captures at least 75% or at least 50% of the FOV of camera 102, for example, at least 75% or at least 50% of the horizontal FOV of camera 102 or at least 75% or at least 50% of the vertical FOV of camera 102. In some embodiments, at least one lens 104 may allow having a working distance (from transparent portion 155) of few centimeters (cm), e.g., 1-5 cm, thus resulting in camera 102, having a field of view (FOV) of few square millimeters (mm), e.g., a FOV of 2×2 mm or 5×3 mm or 2×3 mm. In some embodiments, at least one lens 104 is connected to camera 102, allowing imaging of an object locates on an object plane which includes tubing holders 110.

In some embodiments, camera 102 may be held by holder 108 (e.g., a chassis) capable of adjusting the distance between the at least one lens 104 and the objects in the transparent portion 155. In some embodiments, holder 108 may allow focusing of camera 102 and lens 104 by moving them relative to tubing holders 110 in a direction substantially perpendicular to their object plane. Moving camera 102 and\or lens 104 may be done mechanically (by the operator) or automatically (auto focusing, AF) by a controller (e.g., controller 120 or another controller) based on an image received from camera 102. In another example, holder 108 may allow shifting camera 102 and lens 104 relative to tubing holders 110 in one or two direction(s) parallel to their object plane, to allow selection of camera 102 FOV.

In some embodiments, light source 106 may provide illumination to at least one camera 102. Light source 106 may be a back light illumination source or a front light illumination source. Light source 106 may illuminate in a specific wavelength (e.g., blue, green, red, IR, multispectral, etc.). Light source 106 may illuminate in broadband wavelength (e.g., white light source or a light source is which illuminating in wavelengths of visible light or 300-800 nanometer). In some cases, fluid passing in tube 154 may contain blood traces from patient's body. Light source 106 may illuminate in red (620-750 nm) or deep-red (650-700 nm) or far-red (700-780 nm) or near-infrared (NIR) wavelengths (780-1000 nm), in which blood is partially transparent (has a low absorption coefficient). In some cases, Light source 106 may be limited to wavelength above 600 nanometers (nm) or wavelength above 635 nm wavelength in the range 600 nm-720 nm or wavelength in the range 650 nm-700 nm. In some cases, Light source 106 may have a peak power for a (maximal) wavelength in the range of 600 nm-720 nm or in the range of 650 nm-700 nm. Light source 106 may have several alternative spectrum ranges from the listed above (e.g., white, red, blue, green, deep-red, etc.), which may illuminate simultaneously in some frames and\or alternately in time for some frames. In some cases, light source 106 may be white light source and system 100 may comprise a light filter (not seen in figures) along the optical path which limits the light arriving at sensor 103 to a specific spectrum range or any combination of the listed above (e.g., red, blue, green, deep red, NIR etc.). In all example herein, a light source 106 is tuned to a spectrum range may be defined such that more than 90% of the power of light or more than 80% of the power of light from source 106 originated in the specified spectrum range. In all example herein, a light source is tuned to a wavelength rage may be defined such that the peak (maximal) power wavelength of light from source 106 is in the specified spectrum range.

In some cases, Light source 106 may be continuous (CW). In some cases, Light source 106 may be triggered in synchronization with camera 102 exposure time periods (e.g., light source 106 illuminate during the exposure time of camera 102, and not illuminate while camera 102 is not triggered to expose to light). In some cases, light source 106 may triggered in synchronization with camera 102 exposure and alternate in projected wavelengths with any combination of wavelengths range given above (e.g., some frames are images in white light and some in deep-red light or some of the frames are imaged in red, green or blue light iteratively etc.). For example, light source 106 may be held by holder 108 to allow back or front illumination of camera 102 FOV. Tubing holder(s) 110 may allow gripping of tube 154 and placing tube 154 in the FOV of at least one camera 102.

In some embodiments, at least one camera 102 may be in communication with controller 120, either wired or wirelessly. Controller 120 may process images coming from at least one camera 102 as detailed below. In one example, controller 120 may be integrated with camera 102 in the same unit\box\package, such that all the processing is done within the camera package. Controller 120 may have means for input and output (IO), such as but not limited to: screen, keyboard, mouse, dials, illumination sources, wireless connectivity (e.g., network connectivity, Bluetooth connectivity, Wi-Fi connectivity, etc.) as discussed with respect to FIG. 6 herein below.

In some embodiments, controller 120 is further configured to identify and classify oocytes in images captured by at least one camera 102. According to some embodiments, controller 120 may use controller vision algorithm(s) to detect oocytes in a stream of images (e.g., a video) received from camera 102. For example, the oocyte identification algorithm may include a detection and tracking pipeline, followed by an accurate segmentation which may output statistics and information. According to an example, detection block may identify per image the existence of an oocyte. A tracking block may follow detection block to track an oocyte across adjacent frames, to avoid over counting of the same oocyte multiple times. Detection and tracking algorithms may include some of the following algorithms: finding active frames, finding the size clarity and position of suspected objects, background removal, gaussian mixture models, change detection, frames cross correlation, Kalman filtering and edge detection.

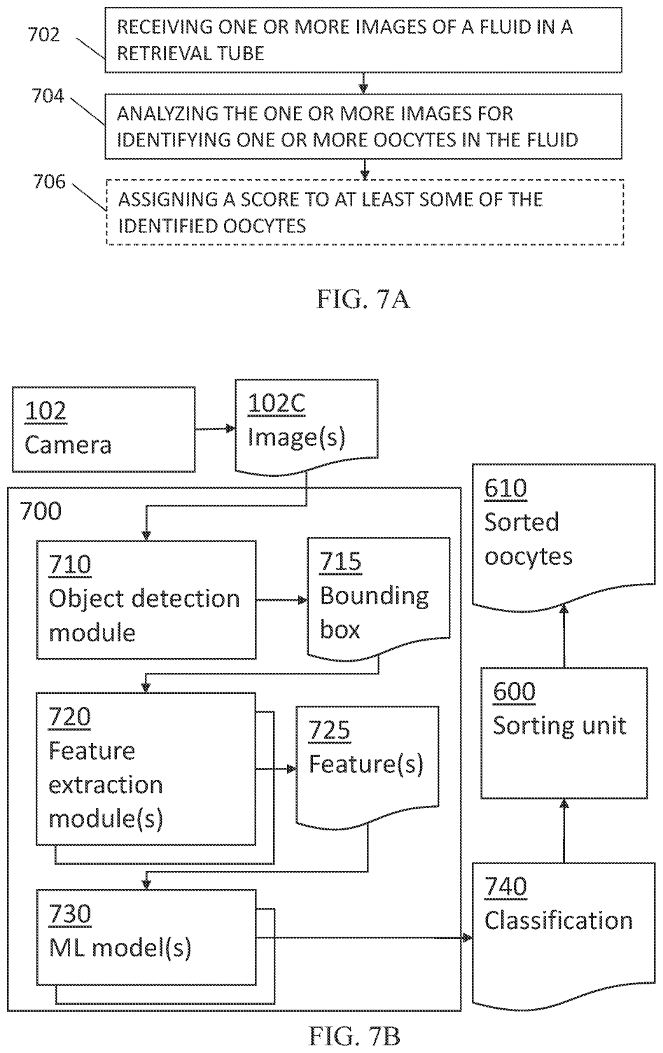

In some embodiments, controller 120 may use a trained ML model for identifying and/or classifying oocytes in the images received from at least one camera 102, as discussed herein below with respect to FIGS. 7A and 7B.

In some embodiments, identifying the oocytes comprises identifying at least one of: number of oocytes, size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity, etc. In some embodiments, controller 120 is further configured to assign a score to at least some of the identified oocytes, for example, based on the listed characteristics.

According to one example, system 100 may show detected oocytes and\or data or grade of detected oocytes to the operator, e.g., on the screen associated with controller 120. Oocyte detection and\or grading may help the operator in decision making during the operation of oocyte retrieval. For example, the doctor may decide to continue or to stop the operation of oocytes retrieval based on the number and grade of oocytes already retrieved.

According to some embodiments, suction unit 156 may be controlled by controller 120. As suction unit create the force that moves oocytes in tube 154 and in and out of the FOV of camera 120, stopping suction in suction unit 156 may stop, delay or move oocytes in the FOV of camera 120. According to one example upon a detection of an oocyte by controller 120, controller may stop suction unit 156 to slow or stop the motion of the oocyte and to take more pictures or pictures at higher exposure time of the oocyte, allowing further examination and scoring of the oocyte. According to one example suction unit 156 may create a force to push oocytes back and forth in the FOV of camera 120.

In some embodiments, system 100 may further include a sorting unit (for example the sorting unit illustrated in FIG. 4) for sorting the fluid flowing in oocytes retrieval tube 154 between at least two different containers 152 and wherein controller 120 is configured to control the sorting unit based on the identification. In a nonlimiting example, the sorting unit may include a plurality of valves, each being in parallel fluid connection with tube 154. In some embodiments, each of the valve may also be in fluid connection with one or more containers (e.g., test tube container 152). In some embodiments, controller 120 may be control at least one valve to open the fluid flow from tube 154 to one of the containers based on analysis of images received from camera 102. For example, if oocytes are identified in the liquid, controller 120 may control a corresponding valve to open and direct the liquid to test tube container 152. If oocytes were not identified in the liquid or that the identified oocytes are of poor quality (e.g., received lower score) controller 120 may control another valve to direct the retrieve fluid into a waste container.

According to some embodiments, system 100 and\or controller 120 may be connected to ultrasound (US) imaging device 160. US imaging device 160 may assist in the operation of oocyte retrieval as known in the art. In one example, US imaging device 160 may be used to assess size, volume, or other quantities of a follicle (containing oocytes) within the patient's ovaries. Assessment of follicle information may be done by means of controller vision or by manual input of the operator. Information from US imaging and\or assessment on follicle quantities may be transferred to controller 120 and added or combined with respective oocytes grading/scoring described herein.

Reference is now made to FIG. 2 which shows another configuration, in which more than one camera (e.g., 2-4) are designed to image tube 154 and oocytes entrained in it simultaneously. FIG. 2 shows perspective view of tube 154 alongside with cameras 102a and 102b with respective, sensors 103a and 103n and lenses 104a and 104b, such that the focal axes of lenes 104a and 104b, marked with 204a and 204 have an angle of 30-180 degrees between them. Adventitiously images from several point of views (POV) may allow the detection of defects in the oocytes from all their circumference. In some embodiments, more-than-one-camera (e.g., 102a and 102b) may be triggered to capture an image simultaneously. In an example, more-than-one-camera (e.g., 102a and 102b) may be triggered to capture images alternately. In an example each camera may be sensitive do a different light wavelength spectrum (e.g., red, green, blue, etc.).

According to one example, seen in FIG. 1, tube 154 is arranged such that its longitudinal dimension is within the focal plane of camera 102.

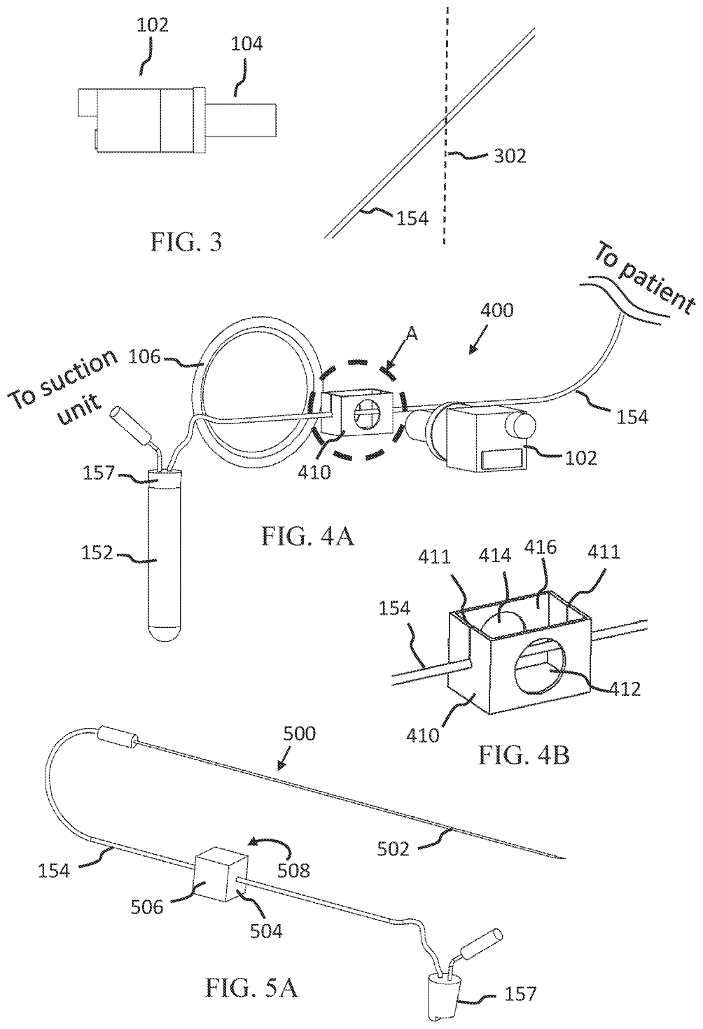

Reference is now made to FIG. 3 which shows another nonlimiting example, in a sideview perspective, of a configuration in which the longitudinal dimension of tube 154 is not with in the focal plane of camera 102. FIG. 3 shows top view of camera 102, lens 104, tube 154 and camera 102 focal plane 302. In some embodiments, there is an angle of 30-60 degrees or 10-30 degrees between the tube 154 longitudinal dimension 304 and camera 102 focal plane 302. In some embodiments, there is an angle of α degrees between the tube 154 longitudinal dimension 304 and camera 102 focal plane 302, where α=ATAN (DOF/HFOV)±10 degrees, and where DOF is the depth of field of camera 102 and HFOV is the horizontal field of view of camera 102 (i.e., along the length of tube 154). According to some embodiments, the flow an oocyte entrained in tube 154 allows for slight focus changes between subsequent frames acquired while the oocyte is in different areas of camera 102 FOV. According to some embodiments, the focus chances may allow finding the frame in which oocytes are in best focus position. According to another example, the focus changes may allow 3D imaging of the oocyte, by combining or fusing plurality of images of the same object. In some embodiments, tube 154 and transparent section 155 may have a circular cross section, which may cause light refractions, and reduction of optical quality of the image.

Reference is now made to FIG. 4A which is an illustration of a system for oocytes retrieval according to some embodiments of the invention. A system 400 may include camera 102, light source 106 and bath 410. Reference is also made to FIG. 4B which is an illustration of an enlarge bath 410, of system 400 according to some embodiments of the invention. In some embodiments, camera 102 and light source may be similar to components described above with regard to system 100 and FIGS. 1-3. In some embodiments, tube transparent section 155 may be inserted into a bath 410. In some embodiments, two slits 411 on the sides of bath may allow the insertion of transparent part 155 into bath 410 while preventing from liquids to leak out of slits 411. In some embodiments, slits 411 may be made of a soft material (e.g., rubber, ethylene-vinyl-acetate, silicone, low-density-polyethylene etc.) which may fill gaps around tube 154 and prevent liquids from passing outside of bath 410. Bath 410 may comprise of a transparent flat front window 412 and a transparent flat back window 414. In some embodiments, both windows 412 and 414 may be made of a transparent material (e.g., glass, acrylic glass (PMMA), silicon, etc.). For example, a color filter such as described above may be integrated into either or both transparent windows 412 and 414 (e.g., to block some portion of the visible light). Front window 412 may allow a line of sight for camera 102 to image transparent part 155. Back window 414 may allow light from light source 106 to enter bath 410 and illuminate section 155. Front window 412 may have flat facets in camera 102 line of sight. In some embodiments, bath 410 may be filled through opening 416 with a material having a refraction index similar to the refraction index of transparent part 155 (in one example, refractive index of filling material is within 10% of the refractive index of transparent part 155, in one example refractive index of filling material is in the range of 1.3-1.6, in one example transparent material may be water or oil, etc.). Imaging of transparent part 155 through flat windows and a bath full of material with refraction index may reduce refraction of the light, increase sharpness of the images, and facilitate oocyte detection or recognition.

Reference is now made to FIG. 5A which is an illustration of an example of a retrieval system according to some embodiments of the invention. Retrieval system 500 may include a needle 502, a transparent tube 154, an optical window 504 and a container cap 157. In some embodiments, needle 502 may be used to penetrate patient body and retrieve oocytes. Needle 502 may be made from a metal (e.g., stainless steel, iron, titanium). Needle 502 be for example 20-60 cm long and have a circular cross section with a diameter of 0.3-2 mm. A lumen in needle 502 (not seen) may be used to create vacuum force and draw oocytes (as known in the art). Oocytes are then passed through tube 154 and via container cap 157 into a container 152 (container 152 not seen, container 152 may or may not be part of system 500). Tube 154 may be made from a soft plastic material (e.g., PVC, TPE, FEP, high-density polyethylene, platinum-cured silicone, and peroxide-cured silicone etc.). Tube 154 may be 0.5-3 meter long, with a cross section circumscribed in a circle having a diameter of 0.5-3 mm. Container cap 157 may also include a port 162 allowing it to connect to a suction unit and create negative pressure in a container like container 152 in order to pull liquids from tube 154. System 500 may further include a viewing window (optical window) 504. Viewing window 504 may be made of a transparent material, e.g., glass, plastic, etc. In one example, viewing window 504 may be made of the same material as tube 154.

In some embodiments, viewing window 504 may be made of a material with refraction index similar to the refraction index of tube 504 (the refraction index of viewing window may be ±10% of the refraction index of tube 154). Viewing window 504 is located on tube 154. Viewing window 504 may allow viewing of the content of tube 154. Viewing window 504 comprise a front flat facet 506. Front flat facet 506 allows light from tube 154 to pass outside with reduced refraction. Front flat facet 506 may have for example an area of 2-20 square mm. In some embodiments, viewing window 154 may further comprise a back flat facet 508. Back flat facet 508 may allow light from external illumination source to pass through tube 508 with reduced refraction.

Reference is now made to FIG. 5B which is an illustration of a usage of system 500 according to some embodiments of the invention. A system 500 may be in use with system 100. In some embodiments, viewing window is located in the FOV of camera 102. For example, light source 106 is located in FOV of camera 102 behind viewing window 504 to allow back illumination. In some embodiments, flat front facet 506 is perpendicular to camara 102 optical axes. In some embodiments, holder (chassis) 108 may be used to hold camera 102, light source 106 and viewing window 504. According to some embodiments, holder 108 may be used to align viewing window 504 relative to camera 102 in all 3 axes (X-Y-Z). In some embodiments, holder 108 may include position pins 520 which may assure the position of viewing window 504 relative to camera 102 within standard deviation of 1 mm in all 3 axes (X-Y-Z) between repetitive positioning experiments.

Reference is now made to FIG. 5C which is an illustration viewing window 504 according to some embodiments of the invention. Viewing window 504 may be made of more than one part (e.g., 2 parts), which may be attached to each other to form a single viewing window 504. The two parts of viewing window 504 may be attached on tube 154. In some embodiments, the two parts of viewing window 504 may be held together mechanically using holder 108. In one example, the two parts of viewing window 504 may be held together and to tube 154 using an optical glue.

Reference is now made to FIG. 6 which shows a nonlimiting example for a sorting unit 600 according to some embodiments of the invention. In some embodiments, sorting unit 600 may be used to sort oocytes and\or follicular fluid in tube 154 into plurality of containers 152. In some embodiments, if a needle is used to aspirate oocytes from follicles in the ovary, the follicular fluid may follow the respective oocyte in tube 154; Thus, sorting of an oocyte into a container may sort its respective follicular fluid into same container. FIG. 6 shows sorting unit 600 in a side view perspective. Sorting unit 600 may or may not be included in system 100. Sorting unit 400 may be controller by controller 120. According to some embodiments, tube 154 may be connected to sorting unit 600 which may include a tube splitter 602 or alike. Tube splitter 602 may split tube 154 into plurality of sublines, each subline may be connected to a container 152, each container is vacuumed by a suction unit (illustrated in FIG. 1). System 600 further includes a series of valves 604 Each subline is further controlled by one valve 604. Valve(s) 604 may be for example solenoid pinch valve or pneumatic valves or ball valves or gate valves, etc. According to an example, valves 604 may be controlled by controller 120 (not seen in figure). By opening and closing valves 604 a selection is made regarding the container 152 to which an oocyte and\or follicular fluid in tube 154 is directed. According to an example, valves 604 are opened and closed based on data or grade extracted from images acquired by camera 102 and processed by controller 120. In some embodiments, each oocyte and\or follicular fluid may be separated into a unique container. In another example, oocytes which have high grade would be separated into one test tube, while oocytes which have low grade would be separated into one other test tube. In another example a user (e.g., medical staff, doctor, nurse, embryologist etc.) may manually decide on the appropriate test tube following an oocyte detection. In some embodiments, system 100 can calculate the speed of oocyte motion in the tube 154. Speed of oocyte motion may be calculated using the translation of the oocyte for sequential images acquired by camera 102. In some embodiments, speed of oocyte motion may be calculated from the level of vacuum force and the viscosity of the fluid medium in the tube. Calculation or measurement of oocyte speed may be used to time sorting mechanism and assure each oocyte identified in the camera FOV arrive at the appropriate container 152.

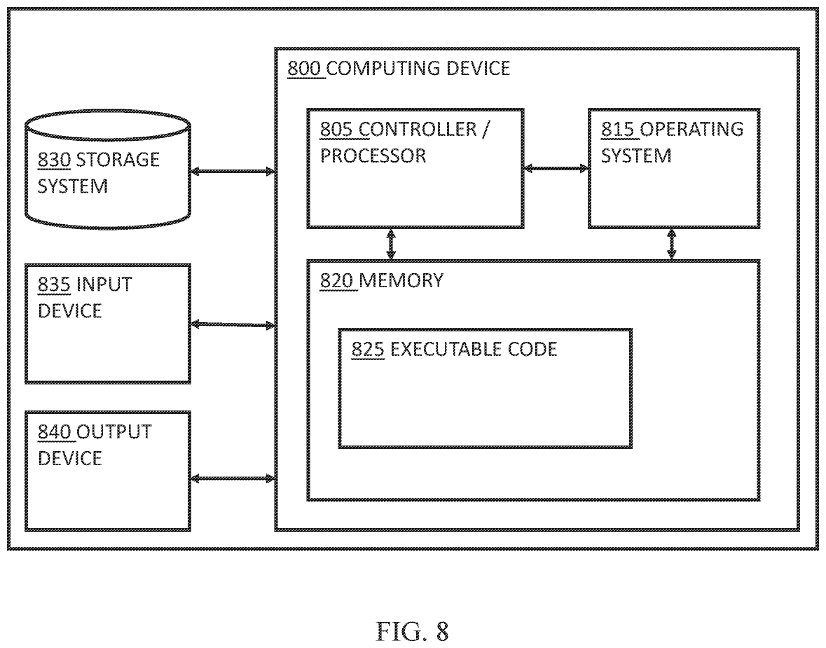

Reference is now made to FIG. 7A which is a flowchart of a method of identifying oocytes in a retrieved fluid, by at least one processor according to some embodiments of the invention. The method of FIG. 7A may be conducted by any processor, for example, controller 120, controller 805 (illustrated and discussed with respect to FIG. 8) or any other suitable processor. In step 702, at least one image of the retrieved fluid may be received from at least one camera 102, 102a and/or 102b. The images may be taken when a liquid that potentially contains oocytes is passing inside oocytes retrieval tube 154 when transparent portion 155 of tube 154 is in the FOV of the camera.

In step 704, the one or more images may be analyzed for identifying one or more oocytes in the fluid. For example, controller 120 may analyze the images using any known methods. For example, controller 120 may use controller vision algorithm(s) to detect oocytes in a stream of images (e.g., a video) received from camera 102.

For example, the oocyte identification algorithm may include a detection and tracking pipeline, followed by an accurate segmentation which may output statistics and information. A tracking block may follow detection block to track an oocyte across adjacent frames, to avoid over counting of the same oocyte multiple times. Detection and tracking algorithms may include some of the following algorithms: finding active frames, finding the size clarity and position of suspected objects, background removal, gaussian mixture models, change detection, frames cross correlation, Kalman filtering and edge detection. In some embodiments, the identification algorithm may include a trained ML model for identifying oocytes in images taken form an oocytes retrieval tube, as discussed with respect to FIG. 7B.

In some embodiments, identifying the oocytes may include identifying and/or scoring at least one of: number of oocytes, size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity.

In step 706, the method may further include assigning a score to at least some of the identified oocytes. Controller 120 may assign the score for each oocyte based on the structure, texture and any other oocytes property that can be received from images analysis. In some embodiments, the score is given based on at least one of: size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity. In some embodiments, data received from US system may be used to add or change the oocyte score.

Reference is now made to FIG. 7B which is a block diagram of a computer software system for classifying oocytes and of using a trained ML model according to embodiments of the present invention.

In some embodiments, a computer software/system 700 may include instruction of a method of classifying oocytes in a retrieved fluid, by at least one processor, for example, controller 120. In some embodiment, at least one image 102C of the retrieved fluid may be received from at least one camera 102 to be processed by system 700. In some embodiments, one or more oocytes may be detected in at least one image 102C, for example, using object detection module 710, using, for example, a bounding box 715 for detecting one or more oocytes in image 102C. Other optional object detection algorithms may include, active frames, finding the size, clarity and/or position of suspected objects, background removal, gaussian mixture models, change detection, frames cross correlation, Kalman filtering and edge detection. In some embodiments, object detection module 510 may be configured to perform the steps of the method of FIG. 7A. Additionally or alternatively, object detection module 510 may include an object detection ML model trained to detect oocytes.

In some embodiments, at least one feature 725 related to the detected one or more oocytes may be extracted from at least one image 102C, using one or more feature extraction modules 720. In some embodiments, the at least one feature related to the oocyte is selected from: number of oocytes, size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity and the like.

In some embodiments, a machine learning (ML) model 730 is applied on the extracted at least one feature 725 to classify the one or more oocytes. In some embodiments, the ML model is trained to classify oocytes based on oocytes quality.

In some embodiments, the classification of one or more oocytes 740 may be sent to controller 120 for controlling system 100. For example, the classification may be used to control storing unit 400 (as illustrated) and/or suction unit 156.

In some embodiments, training ML model 730 may include: receiving a training dataset, comprising a plurality of images 102C, each depicting at least one oocyte and receiving a set of quality labels, corresponding to the plurality of images 102C. In some embodiments, the quality labels may include a score for at least some of the oocytes, determining if the oocyte is suitable for fertilization. In some embodiments, the training may further include, extracting from at least one image of the training dataset at least one respective feature of the depicted at least one oocyte, for example, using feature extraction modules 720; and using the set of quality labels as supervisory data for training the second ML model to classify at least one depicted oocyte based on the extracted features.

Reference is made to FIG. 8, showing a high-level block diagram of an exemplary computing device according to embodiments of the present invention. Computing device 800 may include a controller 805 that may be, for example, a central processing unit processor (CPU), a chip or any suitable computing or computational device, an operating system 815, a memory 820, an executable code 825, a storage 830, input devices 835 and output devices 840. Controller 805 may be configured to carry out methods described herein, and/or to execute or act as the various modules, units, etc. More than one computing device 800 may be included, and one or more computing devices 800 may act as the various components, for example the components shown in FIG. 1. For example, controller 120 described with reference to FIG. 1 above, may be, or may include components of, computing device 800. For example, by executing executable code 825 stored in memory 820, controller 805 may be configured to carry out a method of oocyte retrieval as described with reference to FIG. 7A above. For example, controller 805 may be configured to receive data from imagers (such as cameras 102a and 102b in FIG. 2) and use the input from the imager to control valves (such as valves 604 in FIG. 6A) and/or suction unit (such as suction unit 156 in FIG. 1) as described above.

Operating system 815 may be, or may include any code segment (e.g., one similar to executable code 825 described herein) designed and/or configured to perform tasks involving coordination, scheduling, arbitration, supervising, controlling or otherwise managing operation of computing device 800, for example, scheduling execution of software programs or enabling software programs or other modules or units to communicate. Operating system 815 may be a commercial operating system.

Memory 820 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long term memory unit, or other suitable memory units or storage units. Memory 820 may be or may include a plurality of, possibly different, memory units. Memory 820 may be a controller or processor non-transitory readable medium, or a controller non-transitory storage medium, e.g., a RAM.

Executable code 825 may be any executable code, e.g., an application, a program, a process, task or script. Executable code 825 may be executed by controller 605 possibly under control of operating system 815. For example, executable code 825 may be an application that identify or detect oocytes in images, as further described above. Although, for the sake of clarity, a single item of executable code 825 is shown in FIG. 8, a system according to embodiments of the invention may include a plurality of executable code segments similar to executable code 825 that may be loaded into memory 820 and cause controller 805 to carry out methods according to embodiments of the present invention. For example, units or modules described herein (e.g., controller 120 in FIG. 1) may be, or may include, controller 805 and executable code 825.

Storage 830 may be or may include, for example, a hard disk drive, a floppy disk drive, a Compact Disk (CD) drive, a CD-Recordable (CD-R) drive, a Blu-ray disk (BD), a universal serial bus (USB) device or other suitable removable and/or fixed storage unit. Content may be stored in storage 830 and may be loaded from storage 830 into memory 820 where it may be processed by controller 805. In some embodiments, some of the components shown in FIG. 8 may be omitted. For example, memory 820 may be a non-volatile memory having the storage capacity of storage 830. Accordingly, although shown as a separate component, storage 630 may be embedded or included in memory 820.

Input devices 835 may be or may include a mouse, a keyboard, a touch screen or pad or any suitable input device. It will be recognized that any suitable number of input devices may be operatively connected to computing device 800 as shown by block 835. Output devices 840 may include one or more displays or monitors, speakers and/or any other suitable output devices. It will be recognized that any suitable number of output devices may be operatively connected to computing device 800 as shown by block 840. Any applicable input/output (I/O) devices may be connected to computing device 800 as shown by blocks 835 and 840. For example, a wired or wireless network interface card (NIC), a printer, a universal serial bus (USB) device or external hard drive may be included in input devices 835 and/or output devices 840.

Embodiments of the invention may include an article such as a controller or processor non-transitory readable medium, or a controller or processor non-transitory storage medium, such as for example a memory, a disk drive, or a USB flash memory, encoding, including or storing instructions, e.g., controller-executable instructions, which, when executed by a processor or controller, carry out methods disclosed hereinabove. For example, an article may include a storage medium such as memory 820, controller-executable instructions such as executable code 825 and a controller such as controller 805.

Some embodiments may be provided in a controller program product that may include a non-transitory machine-readable medium, stored thereon instructions, which may be used to program a controller, controller, or other programmable devices, to perform methods as disclosed herein. Embodiments of the invention may include an article such as a controller or processor non-transitory readable medium, or a controller or processor non-transitory storage medium, such as for example a memory, a disk drive, or a USB flash memory, encoding, including or storing instructions, e.g., controller-executable instructions, which when executed by a processor or controller, carry out methods disclosed herein. The storage medium may include, but is not limited to, any type of disk including, semiconductor devices such as read-only memories (ROMs) and/or random access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPU) or any other suitable multi-purpose or specific processors or controllers (e.g., controllers similar to controller 105), a plurality of input units, a plurality of output units, a plurality of memory units, and a plurality of storage units. A system may additionally include other suitable hardware components and/or software components. In some embodiments, a system may include or may be, for example, a personal controller, a desktop controller, a laptop controller, a workstation, a server controller, a network device, or any other suitable computing device. For example, a system as described herein may include one or more devices such as computing device 800.

Unless explicitly stated, the method embodiments described herein are not constrained to a particular order in time or chronological sequence. Additionally, some of the described method elements may be skipped, or they may be repeated, during a sequence of operations of a method.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A system for oocytes retrieval, comprising:
at least one camera;
a holder configured to hold the camera and an oocytes retrieval tube such that a transparent portion of the oocytes retrieval tube is within the field of view (FOV) of the at least one camera; and
a controller configured to:
control the at least one camera to capture images of fluid flowing in the transparent portion;
wherein the transparent portion is transparent to visible light, and the suction unit is configured to suction oocytes in the oocytes retrieval tube.

2. The system of claim 1, wherein the controller is further configured to control a suction unit based on an analysis of the captured images and wherein controlling the suction unit comprises at least one of: terminating the suction, reinitiating the suction and changing the suction velocity.

3. The system of claim 1, wherein the controller is further configured to identify oocytes in the captured images.

4. The system of claim 3, wherein identifying the oocytes comprises identifying at least one of: number of oocytes, size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity.

5. The system of claim 3, wherein controlling the suction unit is based on the identification of the oocytes.

6. The system of claim 3, wherein the controller is further configured to assign a score to at least some of the identified oocytes.

7. The system of claim 6 wherein the score of an identified oocyte is based on at least one of: size of the identified oocyte, shape of the identified oocyte, morphology of the identified oocyte, cytoplasm of the identified oocyte, ooplasm characteristics of the identified oocyte, structure of the perivitelline space, corona radiata quality, zona pellucida, clarity and uniformity.

8. (canceled)

9. The system of claim 3, comprising a sorting unit for sorting the fluid flowing in the oocytes retrieval tube between at least two different containers.

10. The system of claim 9, wherein the controller is configured to control the sorting unit based on the identification.

11. The system of claim 9, wherein the controller is configured to control the sorting unit based on analysis of the images captured by the camera.

12. The system of claim 1, further comprising a light source positioned to provide light to the transparent portion.

13.-18. (canceled)

19. A method of oocytes retrieval, comprising:
receiving one or more images of a fluid in a retrieval tube; and
analyzing the one or more images for identifying one or more oocytes in the fluid.

20. The method of claim 19, wherein identifying the oocytes comprises identifying at least one of: number of oocytes, size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity.

21. The method of claim 19, further comprising assigning a score to at least some of the identified oocytes.

22. The method of claim 21, wherein the score is given based on at least one of: size of an oocyte, shape of an oocyte, morphology of an oocyte, cytoplasm of an oocyte, ooplasm characteristics of an oocyte, structure of the perivitelline space, corona radiata quality, zona pellucida, clarity and uniformity.

23. The method of claim 21, further comprising:
sorting the fluid flowing in the oocytes retrieval tube between at least two different containers.

24. A method of classifying oocytes in a retrieved fluid, by at least one processor, said method comprising:
receiving at least one image of the retrieved fluid from at least one camera;
detecting one or more oocytes in the at least one image;
extracting from the at least one image at least one feature related to the detected one or more oocytes;
applying a ML model on the extracted at least one feature to classify the one or more oocytes,
wherein said ML model is trained to classify oocytes based on oocytes quality.

25. The method of claim 24, wherein training the ML model comprises:
receiving a training dataset, comprising a plurality of images, each depicting at least one oocyte;
receiving a set of quality labels, corresponding to the plurality of images;
extracting from at least one image of the training dataset at least one respective feature of the depicted at least one oocyte; and
using the set of quality labels as supervisory data for training the ML model to classify at least one depicted oocyte based on the extracted features.

26.-30. (canceled)
